(19) United States
(12) Patent Application Publication
(10) Pub. No.: US 2006/0258738 A1
(43) Pub. Date: Nov. 16, 2006

(54) Use of dronabinol for treatment of side effects of Hepatitis C therapy

(76) Inventor: Douglas Dieterich, Garden City, NY (US)

Correspondence Address: Janet M. MacLeod;DORSEY & WHITNEY LLP, Intellectual Property Department, 250 Park Avenue, New York, NY, 10177, US

(21) Appl. No.: 11/433,378

(22) Filed: May 12, 2006

Related U.S. Application Data
(60) Provisional application No. 60/680,952, filed on May 12, 2005.

(51) Int. Cl. A61K 31/353; A61K 31/7056; A61K 38/21

(52) U.S. Cl. 514/454; 514/43; 424/85.4

(57) ABSTRACT

The present invention provides methods for treating and preventing the gastrointestinal side effects associated with anti-HCV therapy comprising administering delta-9-tetrahydrocannabinol.

### CROSS REFERENCE TO RELATED APPLICATION

This application claims the priority of U.S. application Ser. No. 60/680,952 filed May 12, 2005, the disclosure of which is incorporated herein by reference.

### BACKGROUND OF THE INVENTION

The compound delta-9-tetrahydrocannabinol (delta-9-THC) is a naturally occurring component of Cannabis sativa L. (marijuana). The synthetic form of delta-9-THC, dronabinol, is approved in the United States for use in the treatment of anorexia associated with weight loss in patients with HIV/AIDS. Such treatment is described, for example, in U.S. Pat. No. 6,703,418. Dronabinol is also approved for use in selected patients for the prevention of nausea and vomiting associated with emetogenic cancer chemotherapy when other antiemetic medications are not effective. Dronabinol is marked in the United States as oral soft gelatin capsules under the trade name Marinol™. Physicians' Desk Reference, 2005, 59^{th} edition, Thompson PDR, Montvale, N.J.

The mode of action of dronabinol is not fully understood, and several mechanisms have been proposed. Grotenhermen (2004) Neuroendocrinology Letters 25:14-23. The majority of dronabinol's effects are thought to be mediated through agonistic actions at cannabinoid receptors, of which two (CB₁ and CB₂) have been identified. Pertwee (1997) Pharmacol. Ther. 74:129-80.

Chronic Hepatitis C Virus (HCV) infection affects approximately 200 million people worldwide, including 3.9 million people in the U.S. NIH Consensus Development Conference Statement: Management of Hepatitis C:2002. Hepatology (2000) 36:S3-S20. Anemia, neutropenia, psychiatric side effects and gastrointestinal (GI) side effects of therapy are common, may be difficult to manage, and constitute major causes of dose reduction or discontinuation of anti-HCV therapy. Early and proactive management of treatment-limiting adverse events and symptoms are the key to improving chances of HCV eradication.

Methods of treating the hematologic effects caused by combined RBV and IFN treatment of HCV are known. The use of erythropoietin for treatment-induced anemia is disclosed by U.S. Pat. No. 6,833,351 and Dieterich et al. (2003) Am. J. Gastroenterol. 98:2491-2499. The use of G-CSF for treatment of severe chronic neutropenia is also known. Physicians' Desk Reference, supra. Pre-emptive anti-depressant therapy is effective for the treatment of INF-induced depression. Musselman et al. (2001) N. Engl. J. Med. 344:961-966.

Gastrointestinal side effects including nausea, vomiting, anorexia and weight loss are frequently experienced by individuals receiving combined RBV and IFN treatment, and may result in significant morbidity and reduction in quality of life. Nausea in up to 35% of patients, vomiting in up to 20% of patients and weight loss of greater than 5 kilograms in up to 10% of patients have been reported. Manns et al. (2001) Lancet 358:958-965; Fried et al. (2002) N. Eng. J. Med. 347:975-982. It has been surprisingly discovered in accordance with the present invention that delta-9-THC is useful for the treatment of GI side effects associated with anti-HCV treatment.

### SUMMARY OF THE INVENTION

The present invention provides a method for treating HCV comprising administering ribavirin (RBV) and interferon (IFN) to a patient infected with HCV and further comprising administering delta-9-tetra hydrocannabinol concomitantly or sequentially with RBV and IFN.

The present invention further provides a method for treating or preventing gastrointestinal side effects induced by anti-HCV therapy comprising administering delta-9-tetra hydrocannabinol to a patient undergoing anti-HCV therapy in an amount effective to treat or prevent such gastrointestinal side effects.

In another embodiment, the present invention provides a kit containing RBV, IFN and delta-9-tetrahydrocannabinol.

### BRIEF DESCRIPTION OF THE DRAWINGS

FIG. 1 is a graph showing the change in body mass index (BMI) relative to baseline in patients receiving dronabinol for gastrointestinal side effects related to anti-HCV therapy.

### DETAILED DESCRIPTION OF THE INVENTION

The present invention provides a method of treating or preventing gastrointestinal side effects of anti-HCV therapy comprising administering delta-9-THC to a patient in need of such treatment or prevention. Anti-HCV therapy is defined herein as a course of interferon (IFN) preferably combined with ribavirin (RBV). The combination of IFN and RBV is standard treatment for HCV-infected patients. Booth et al. (2001) Gut 49:Suppl 1: I-1; Lai et al. (1996) Gastroenterology 111:1307-1312; Fried et al. (2002) N. Engl. J. Med. 347:975-982. In a preferred embodiment the IFN is pegylated IFN. In another preferred embodiment the pegylated IFN peginterferon α-2b or α-2a. Manns et al. (2001) Lancet 358:958-965; Zu et al. (1998) Hepatology 28:702A. Regimens for such combination therapy are well-known to those of ordinary skill in the art. For example, RBV is typically provided in solid form as 1000-1200 mg/daily in two divided doses. IFN may be provided in injectable form as a 3 MIU dose administered three times weekly. Dosing and administration may be optimized by those of ordinary skill in the art depending upon a patient's age, weight, health, HCV genotype, viral load, and so on. Hadziyannis et al. (2004) Ann. Intern. Med. 140:346. RBV is available as Copegus™ (Hoffmann-LaRoche, Nutley, N.J.; manufacturer's recommended dose of 800-1200 mg. p.o. daily in two divided doses, dose depending upon patient weight) and Rebetol™ (Schering-Plough Corporation, Kenilworth, N.J.; manufacturer's recommended dose of 800 mg. p.o. daily in two divided doses). See Physicians' Desk Reference, supra. Pegylated INF α-2a is available as Pegasys™ (Hoffmann-LaRoche; manufacturer's recommended dose of 180 μg s.c. once weekly). Pegylated INF α-2b is available as Peg-lntron™ (Schering-Plough Corporation; manufacturer's recommended dose of 1.5 μg/kg s.c. once weekly). Id.

The invention further provides a method for treating HCV comprising administering RBV and IFN to a patient infected with HCV and further comprising administering delta-9-THC to the patient. In one embodiment the patient is co-infected with HIV. In another embodiment the patient is not co-infected with HIV.

Gastrointestinal side effects of anti-HCV therapy include nausea, vomiting, anorexia and weight loss. The term “treatment of gastrointestinal side effects” as used herein means the amelioration of at least one of nausea, vomiting, anorexia and weight loss. Amelioration may be measured subjectively, for example by a patient's report of improvement of symptoms of nausea, vomiting or anorexia, or objectively, for example as a lack of change or an increase in body mass index (BMI) at the end of treatment compared to baseline.

The term “prevention of gastrointestinal side effects” as used herein refers to a statistically significant reduction in symptoms of at least one of nausea, vomiting, anorexia and weight loss relative to a control population of patients undergoing anti-HCV therapy and not receiving delta-9-THC.

In a preferred embodiment, the delta-9-THC is synthetic delta-9-THC, also known as dronabinol. Dronabinol is available as Marinol™ (Unimed Pharmaceuticals, Inc., Marietta, Ga.) in the form of soft gelatin capsules for oral administration. Id. Other formulations of dronabinol may also be administered in the method of the present invention, including for example dronabinol formulated for inhalation such as by deep long aerosol or nasal spray, or as a nasal gel, sublingual preparation or suppository. Dronabinol may be administered orally, by injection, or by intranasal, transdermal, inhalation, sublingual or rectal administration. In a preferred embodiment, dronabinol is administered orally.

Dronabinol is administered in an amount effective to treat or prevent at least one gastrointestinal side effect of anti-HCV treatment. Those of ordinary skill in the art can determine an effective amount of dronabinol with reference to the patient's weight, health, use of other therapy for gastrointestinal symptoms, and the route of administration of dronabinol. In a preferred embodiment, dronabinol is administered orally at a dose of 1.0 mg to 50 mg per day. In a more preferred embodiment dronabinol is administered orally at a dose of 2.5 mg to 30 mg per day. In a most preferred embodiment, dronabinol is administered orally at a dose of 10 mg to 20 mg per day. Dronabinol may be administered in a single dose or in equally divided doses over a twenty four hour period, for example every four to six hours. Those of skill in the art can determine an optimal dose by initiating therapy at a low dose and titrating until amelioration of symptoms is achieved.

Dronabinol administration may be commenced before, during or after administration of IFN and RBV, and may proceed concomitantly and/or sequentially with administration of IFN and RBV. Dronabinol, IFN and RBV may be co-administered, i.e., administered at or about the same time. Dronabinol may be administered for from one day to several months, depending upon the duration of anti-HCV therapy and the severity of gastrointestinal side effects. Those of skill in the art can determine the appropriate time to commence administration relative to anti-HCV therapy, and the optimal duration of treatment. The dose of dronabinol may be adjusted during the course of administration in order to minimize adverse effects and optimize alleviation of gastrointestinal side effects of anti-HCV treatment.

The present invention further provides a kit containing RBV, IFN and delta-9-THC. In a preferred embodiment, IFN is pegylated IFN and delta-9-THC is dronabinol. In a more preferred embodiment, dronabinol is provided as a soft gelatin capsule. The kit preferably comprises IFN formulated for injection and RBV and delta-9-THC each formulated for oral administration. The kit may further comprise devices for administration by injection, such as syringes. The kit may further comprise instructions for administration of IFN, RBV and delta-9-THC.

All references cited herein are incorporated herein in their entirety.

The following non-limiting examples serve to further illustrate the invention.

### EXAMPLE 1

The following example demonstrates the efficacy and tolerability of dronabinol for the treatment of nausea/vomiting, anorexia and weight loss in a cohort of chronic HCV patients treated with IFN and RBV.

Methods

A retrospective chart review of 22 patients with chronic HCV receiving IFN (Pegylated n=18, standard n=4) and RBV was performed. All were receiving open label dronabinol for specific GI side effects (nausea/vomiting, anorexia and weight loss) related to anti-HCV therapy. Over half the cohort (13/22) were co-infected with HIV. Demographic, clinical, and virological data was recorded at baseline (pre dronabinol therapy); Body Mass Index (BMI) was calculated at baseline, and at the end of dronabinol therapy.

A positive response to dronabinol was defined as follows:

Subjective: Documentation in chart of improvement in symptoms of nausea, vomiting or anorexia, as reported by patient.

Objective: No change or increase in BMI at end of dronabinol treatment compared to baseline.

Results

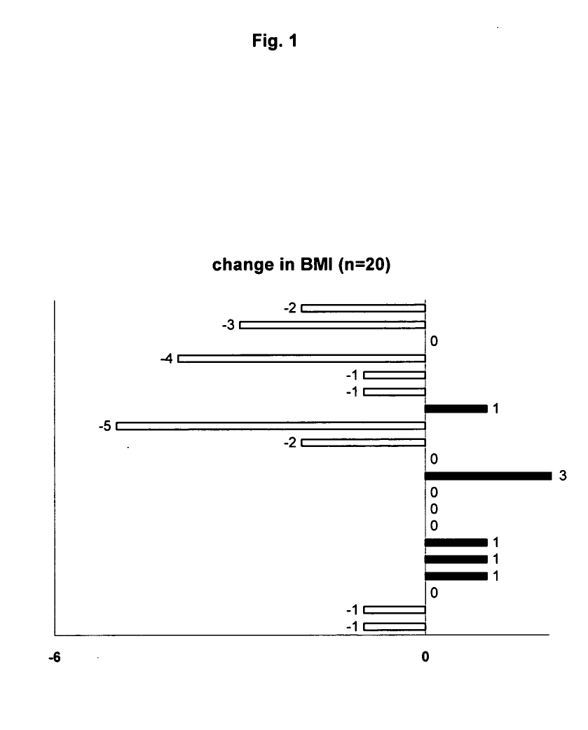

Baseline demographic, virological and clinical details of the cohort are summarized in Table 1. Change in BMI at end of therapy compared to baseline is shown in FIG. 1.

TABLE 1

| | | | |
|---|---|---|---|
| Age (years) | 46 | (33-62) | median (range) |
| Male sex | 17/22 | (77) | n (%) |
| Former IVDU | 9/22 | (41) | n (%) |
| Current cannabis user | 4/22 | (18) | n (%) |
| HCV genotype 1 | 20/22 | (91) | n (%) |
| HCV VL > 2 × 10\hat{ }6 cps/ml | 13/21 | (62) | n (%) |
| Biopsy stage 3 or 4/4 | 6/20 | (30) | n (%) |
| BMI kg/m\hat{ }2 | 24.5 | (18-43) | median (range) |
| HIV co-infected | 13/22 | (59) | n (%) |
| Absolute CD4 × 10\hat{ }6/L | 430 | (162-945) | median (range) |
| on ART | 13/13 | (100) | n (%) |

Indications for Dronabinol Therapy

The commonest indications were nausea/vomiting alone (7/22) and the combination of anorexia and weight loss (7/22). Four patients received dronabinol for anorexia, one individual for nausea alone and one for nausea/vomiting, anorexia and weight loss.

The median (maximum) dose of dronabinol used was 10 mg daily (range 2.5 to 30 mg). Dronabinol was used as first line therapy in 13/22 (59%) and as an adjunct to existing therapy for GI symptoms in 9/22 (41%).

Adverse Events

Dronabinol was well tolerated, with only 3 patients (14%) developing adverse effects, the commonest being CNS toxicity. Two patients discontinued dronabinol, one because of symptoms suggestive of depersonalization, the other discontinuation was due to worsening nausea. One individual ascribed dizziness and hyperactivity to dronabinol but elected to continue the medication.

Outcome of Dronabinol Therapy

Using the definitions described earlier, a positive subjective outcome was observed in 10/22 (45%) of the cohort, and 11/20 (55%) experienced a positive objective outcome, see Table 2.

TABLE 2
Positive response to dronabinol therapy

| | Symptoms | BMI |
|---|---|---|
| Whole cohort | 10/22 (45%) | 11/20 (55%) |
| HIV | 5/13 (38%) | 8/13 (62%) |
| Former IVDU | 5/9 (55%) | 4/8 (50%) |
| Current cannabis | 2/4 (50%) | 3/4 (75%) |

Among the 13 HIV co-infected individuals, 5 reported symptomatic relief with dronabinol, and assessment of BMI showed that 8/13 experienced stabilization or increase in BMI.

Of the former IV drug users in the cohort, over 50% (5/9) reported improvement of symptoms with dronabinol and nearly half (4/9) objectively demonstrated a positive response. Although there were a limited number of patients who were known to be current marijuana users, it is notable that 2/4 (50%) reported a positive response to dronabinol therapy subjectively, and 3/4/ (75%) demonstrated a positive objective response. Dronabinol was discontinued due to lack of efficacy in 8/22 (35%).

### CONCLUSIONS

Individuals receiving anti-HCV therapy frequently experience GI side effects, which can persist throughout the course of treatment and may result in significant morbidity and reduction in quality of life, particularly in HIV/HCV co-infected patients. In this cohort, a positive outcome with dronabinol was observed in over 40% of the cohort, both for improvement in symptoms and stabilization or increase in BMI. Dronabinol was well-tolerated by the majority of the cohort, including those with HIV/HCV, discontinuation due to adverse effects being noted in just 2/22 (9%). Both discontinuations occurred amongst the 9 mono-infected HCV patients. Of interest, dronabinol was also well tolerated in the 4 members of the cohort who were aged 60 or greater.

1. A method of treating or preventing gastrointestinal side effects of anti-Hepatitis C Virus (anti-HCV) therapy comprising administering delta-9-tetrahydrocannabinol (delta-9-THC) to a patient in need of such treatment or prevention.

2. The method of claim 1 wherein the patient is undergoing treatment of HCV with interferon.

3. The method of claim 1 wherein the patient is undergoing treatment of HCV with interferon and ribavirin.

4. The method of claim 1 wherein said delta-9-THC is dronabinol.

5. The method of claim 1 wherein said delta-9-THC is administered by oral, intranasal, transdermal, sublingual or rectal administration, injection or inhalation.

6. The method of claim 1 wherein said delta-9-THC is administered by oral administration.

7. The method of claim 4 wherein said dronabinol is administered orally at a dose of 1.0 mg to 50 mg per day.

8. The method of claim 4 wherein said dronabinol is administered orally at a dose of 2.5 mg to 30 mg per day.

9. The method of claim 4 wherein said dronabinol is administered orally at a dose of 10 mg to 20 mg per day.

10. A method of treating Hepatitis C Virus (HCV) comprising administering interferon and ribavirin to a patient infected with HCV and further comprising administering delta-9-THC to said patient.

11. The method of claim 10 wherein said patient is co-infected with HIV.

12. The method of claim 11 wherein said patient is not co-infected with HIV.

13. The method of claim 10 wherein said delta-9-THC is dronabinol.

14. The method of claim 10 wherein said delta-9-THC is administered by oral, intranasal, transdermal, sublingual or rectal administration, injection or inhalation.

15. The method of claim 10 wherein said delta-9-THC is administered by oral administration.

16. The method of claim 13 wherein said dronabinol is administered orally at a dose of 1.0 mg to 50 mg per day.

17. The method of claim 13 wherein said dronabinol is administered orally at a dose of 2.5 mg to 30 mg per day.

18. The method of claim 13 wherein said dronabinol is administered orally at a dose of 10 mg to 20 mg per day.

19. A kit containing RBV, IFN and delta-9-THC.

20. The kit of claim 19 wherein IFN is formulated for injection and RBV and delta-9-THC are each formulated for oral administration.

21. The kit of claim 19 wherein delta-9-THC is dronabinol.

22. The kit of claim 21 wherein dronabinol is formulated as a soft gelatin capsule.
